(19) United States
(12) Patent Application Publication
(10) Pub. No.: US 2026/0263541 A1
(43) Pub. Date: Sep. 10, 2026

(54) USE OF LACTIPLANTIBACILLUS PLANTARUM GOLDGUT-HNU082 IN IMPROVING GUT HEALTH IN MIDDLE-AGED AND ELDERLY PEOPLE

(71) Applicant: SHENZHEN PORSHEALTH BIOENGINEERING CO., LTD., SHENZHEN (CN)

(72) Inventors: JIE PAN, SHENZHEN (CN); XIAOMIN XU, SHENZHEN (CN); JUN WANG, SHENZHEN (CN); SILU ZHANG, SHENZHEN (CN); LE LIU, SHENZHEN (CN); GUOXUN XIAO, SHENZHEN (CN)

(21) Appl. No.: 19/357,615

(22) Filed: Oct. 14, 2025

(30) Foreign Application Priority Data
Mar. 6, 2025 (CN) 202510259154.0

(51) Int. Cl. A61K 35/747; A61P 1/00; A61P 3/00; A61P 3/10

(52) U.S. Cl. CPC A61K 35/747; A61P 1/00; A61P 3/00; A61P 3/10

(57) ABSTRACT

Uses of Lactiplantibacillus plantarum GOLDGUT-HNU082 in improving gut health in middle-aged and elderly people, preparing a bacterial agent for improving gut health, preparing a product for increasing GMWI2, and preparing a product for lowering blood glucose are provided, relating to the field of microbial technology. The deposit number of Lactiplantibacillus plantarum GOLDGUT-HNU082 is CGMCC NO. 33075.

### CROSS REFERENCE TO RELATED APPLICATIONS

The present disclosure claims the priority to the Chinese patent application with the filing No. 202510259154.0, entitled “USE OF LACTIPLANTIBACILLUS PLANTARUM GOLDGUT-HNU082 IN IMPROVING GUT HEALTH IN MIDDLE-AGED AND ELDERLY PEOPLE” and filed on Mar. 6, 2025 with the Chinese Patent Office, the contents of which are incorporated herein by reference in their entirety.

### TECHNICAL FIELD

The present disclosure relates to the field of microbial technology, and more particularly to a use of Lactiplantibacillus plantarum GOLDGUT-HNU082 in improving gut health of middle-aged and elderly people.

### BACKGROUND ART

As China has officially entered a moderately aging society, promoting the health of the elderly as a collective effort has become an important national strategy. Over 180 million elderly people in China currently suffer from various chronic diseases, such as hypertension and diabetes. With the increase of age, the physiological metabolic functions and immune functions of various tissues and organs in these middle-aged and elderly people gradually decline, significantly increasing the risk of suffering from viral or bacterial infections. Although no obvious pathological changes are observed in the clinical indicators of some middle-aged and elderly people, their health may still deteriorate towards sub-health, primarily due to the potential hidden impact of gut health.

A vast number of microorganisms of diverse species inhabit the human gastrointestinal tract, and these microorganisms are collectively referred to as the gut microbiota. Numerous studies have indicated that the gut microbiota is associated with the health of middle-aged and elderly people, where its balance and diversity may influence such aspects as the immune system, metabolism, and inflammatory responses of human body, among other aspects. Dysbiosis of the gut microbiota may lead to damage to the intestinal mucosal barrier and an increase in intestinal inflammatory factors, which in turn may contribute to the development of major chronic diseases of the immune, metabolic, and digestive systems in the elderly.

The Gut Microbiome Wellness Index (GMWI2) assesses health status based on species-level taxonomy of fecal metagenomic samples. The GMWI2 index is determined by comparing the relative abundance of two groups of microbial species associated with favorable and unfavorable health statuses, and has been reported to be capable of distinguishing between gut-healthy and gut-unhealthy populations. The GMWI2 was also applied in four recently published longitudinal gut microbiome studies, demonstrating its broad applicability for tracking gut health. These studies encompassed fecal microbiota transplantation (FMT) therapy, dietary interventions, antibiotic treatment, and the effects of prebiotic oligosaccharides on gut microbial communities. The results demonstrate that the GMWI2 may reflect changes in gut health earlier than traditional diversity indices, providing a valuable tool for assessing gut microbiome recovery after acute illness, the impact of dietary fiber on gut health, and other such aspects.

Although there is an increasing number of studies on gut microbiota, few studies use specific indicators to measure the overall health level of the gut microbiota, and there are also limited microbial resources that may clearly improve the gut microbial health index.

Therefore, discovering more microbial resources that improve gut health to collectively promote the health of the elderly is an urgent issue that needs to be addressed by the person skilled in the art.

### SUMMARY

In view of this, the present disclosure provides a use of Lactiplantibacillus plantarum GOLDGUT-HNU082 in improving gut health of middle-aged and elderly people.

In order to achieve the above purpose, the present disclosure adopts the following technical solutions:

the use of Lactiplantibacillus plantarum GOLDGUT-HNU082 in improving gut health, where the deposit number of Lactiplantibacillus plantarum GOLDGUT-HNU082 is CGMCC NO. 33075.

Another purpose of the present disclosure is to provide a use of Lactiplantibacillus plantarum GOLDGUT-HNU082 in the preparation of a bacterial agent (probiotic) for improving gut health, where the deposit number of the Lactiplantibacillus plantarum GOLDGUT-HNU082 is CGMCC NO. 33075.

Another purpose of the present disclosure is to provide a use of a bacterial agent containing Lactiplantibacillus plantarum GOLDGUT-HNU082 in the preparation of a product for improving gut health, where the deposit number of Lactiplantibacillus plantarum GOLDGUT-HNU082 is CGMCC NO. 33075.

Another purpose of the present disclosure is to provide a use of Lactiplantibacillus plantarum GOLDGUT-HNU082 in the preparation of a product that increases GMWI2, where the deposit number of Lactiplantibacillus plantarum GOLDGUT-HNU082 is CGMCC NO. 33075.

Another purpose of the present disclosure is to provide a use of Lactiplantibacillus plantarum GOLDGUT-HNU082 in the preparation a product for lowering blood glucose, where the deposit number of the Lactiplantibacillus plantarum GOLDGUT-HNU082 is CGMCC NO. 33075.

Another purpose of the present disclosure is to provide a use of Lactiplantibacillus plantarum GOLDGUT-HNU082 in the preparation of a product for reducing serum total protein, where the deposit number of the Lactiplantibacillus plantarum GOLDGUT-HNU082 is CGMCC NO. 33075.

Beneficial effects: the Lactiplantibacillus plantarum GOLDGUT-HNU082 of the present disclosure significantly improves the gut health of the middle-aged and elderly population, increases the GMWI2 index, and enhances the microbial abundance of beneficial bacteria; and it also has a lowering effect on subjects with high fasting blood glucose levels and may improve the subjects' liver function-related indicators.

### BRIEF DESCRIPTION OF DRAWINGS

In order to more clearly illustrate the technical solutions of the embodiments of the present disclosure or in the prior art, the drawings required for use in description of the embodiments or the prior art will be briefly introduced below. Obviously, the drawings described below are merely embodiments of the present disclosure. For a person ordinarily skilled in the art, other drawings may be obtained based on the provided drawings without paying any creative work.

FIG. 1 is a colony morphology diagram of Lactiplantibacillus plantarum GOLDGUT-HNU082.

FIG. 2 shows the whole genome comparison results of Lactiplantibacillus plantarum GOLDGUT-HNU082.

FIG. 3 shows the test results of serum biochemical indicators before and after taking Lactiplantibacillus plantarum GOLDGUT-HNU082.

FIG. 4A shows the changes in the gut health index before and after taking Lactiplantibacillus plantarum GOLDGUT-HNU082, FIG. 4B shows the Shannon index and Simpson index of the gut microbiome before and after taking Lactiplantibacillus plantarum GOLDGUT-HNU082, and FIG. 4C shows the principal coordinate analysis results of the Bray-Curtis distance before and after taking Lactiplantibacillus plantarum GOLDGUT-HNU082.

FIG. 5A shows the gut microbiome annotation results at the phylum level, and genus level before and after taking Lactiplantibacillus plantarum GOLDGUT-HNU082, and FIG. 5B and FIG. 5C show the gut microbiome annotation results at species level before and after taking Lactiplantibacillus plantarum GOLDGUT-HNU082.

FIG. 6A shows the metabolic pathway analysis results before and after taking Lactiplantibacillus plantarum GOLDGUT-HNU082, and FIG. 6B shows the correlation analysis results before and after taking Lactiplantibacillus plantarum GOLDGUT-HNU082.

### DETAILED DESCRIPTION OF EMBODIMENTS

The technical solutions in the embodiments of the present disclosure will be clearly and completely described below in conjunction with the drawings in the embodiments of the present disclosure. Obviously, the described embodiments are only some of the embodiments of the present disclosure, not all of the embodiments. Based on the embodiments of the present disclosure, all other embodiments obtained by a person ordinarily skilled in the art without making creative efforts are within the scope of protection of the present disclosure.

### Example 1

Isolation, screening and identification of Lactiplantibacillus plantarum GOLDGUT-HNU082

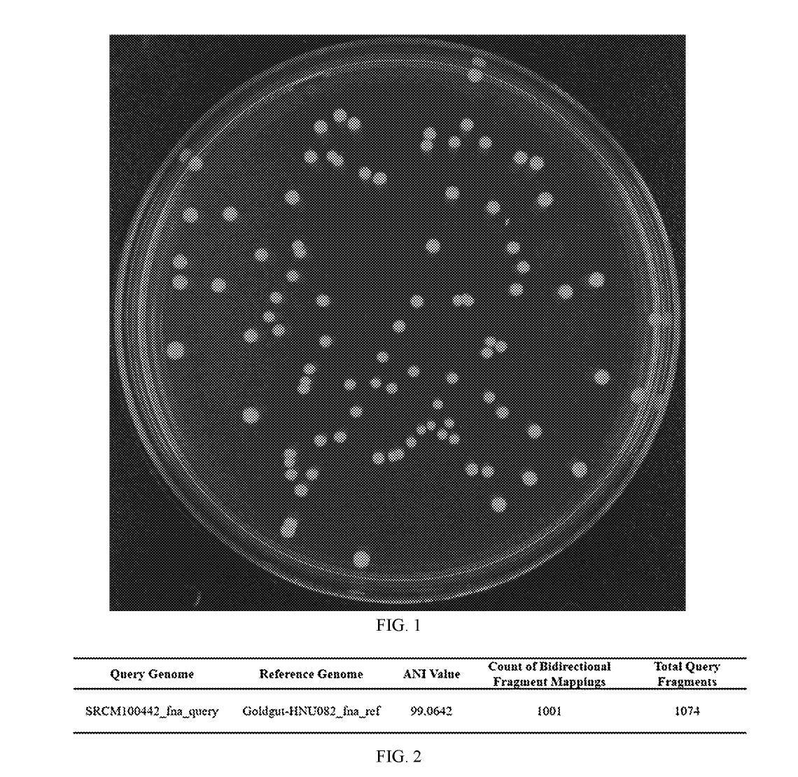

1. 30 fermented food samples were collected for subsequent use from farmers' markets in various counties and cities in Hainan Province, including Wanning, Wenchang, Wuzhishan, Baisha, and Lingshui, etc. The collected fermented food samples were diluted tenfold in sterilized physiological saline and subsequently prepared into sample dilutions at gradients of: 10⁻¹, 10⁻², 10⁻³, 10⁻⁴, and 10⁻⁵ by using a ten-fold dilution method. 0.1 mL of 10⁻³, 10⁻⁴, and 10⁻⁵ sample dilutions (sample dilutions at the gradients of 10⁻³, 10⁻⁴, and 10⁻⁵) were respectively spread onto a MRS agar medium, cultured anaerobically at 37°C for 48 h, and subjected to multiple times of isolation, streaking, purification, culturing, and identification, to screen out 5 Lactiplantibacillus plantarum strains. The 5 strains were cultured in a MRS liquid for 24 h, and the OD value and viable bacteria count were measured, and finally, a Lactiplantibacillus plantarum strain was screened out and named GOLDGUT-HNU082. The purified GOLDGUT-HNU082 strain was inoculated into a MRS broth medium and activated for 24 h, and then spread onto a MRS agar medium, and after culturing at 37 °C for 24 h, the bacterial colony was white, had a diameter of 0.1-0.2 cm, and had a smooth, moist, raised surface and a neat edge (as shown in FIG. 1). When observed under an optical microscope, the bacteria were rod-shaped.

2. Identification Information: the GOLDGUT-HNU082 strain was subjected to whole genome sequencing. Sequences obtained from whole genome sequencing were subjected to genomic alignment. Genomic similarity analysis was performed by using the online analysis platform Galaxy (https://usegalaxy.org/) and FastANI software (https://github.com/ParBLiSS/FastANI). The analysis results showed that the ANI value of the whole genome sequence of GOLDGUT-HNU082 and that of Lactiplantibacillus plantarum SRCM100442 was 99.0642 (as shown in FIG. 2).

3. The strain was deposited in the China General Microbiological Culture Collection Center (CGMCC), with the deposit address at the Institute of Microbiology, Chinese Academy of Sciences, No. 3, Yard 1, Beichen West Road, Chaoyang District, Beijing. The deposit number is CGMCC NO. 33075, the deposit date is December 16, 2024, and the taxonomic name is Lactiplantibacillus plantarum.

### Example 2

Lactiplantibacillus plantarum GOLDGUT-HNU082 improving gut health

1. Subject recruitment

We enrolled 14 healthy middle-aged and elderly subjects (aged 40–71 years). All participants underwent a comprehensive physical examination. Exclusion criteria included: 1. a history or family history of colon cancer, celiac disease, or inflammatory bowel disease; 2. previously diagnosed with organic intestinal disease by colonoscopy; 3. Individuals preparing for pregnancy (male and female), or pregnant or lactating women; 4. people who are allergic to the sample or its ingredients; 5. use of probiotics or probiotic-related preparations within the past two weeks; 6. use of anti-anxiety, antidepressant, or other psychotropic medications within the past two weeks; 7. previously diagnosed with serious illness, such as myocardial infarction, cerebral infarction, or malignant tumor, deemed inappropriate for enrollment by the investigators; 8. subjects with significant mental illnesses who have difficulty in controlling their actions and are unable to cooperate; and 9. subjects who are illiterate and unable to understand informed consent form or sign the informed consent form.

2. Lactiplantibacillus plantarum GOLDGUT- HNU082 intervention

Subjects were instructed to consume 2 grams of Lactiplantibacillus plantarum GOLDGUT-HNU082 (containing 1.0 × 10¹⁰ CFU/g of viable bacteria) daily for 60 consecutive days. Participants were required to provide fecal and serum samples before taking probiotics (T0 period) and 60 days after taking probiotics (T1 period). There were no dietary restrictions during the intervention period.

3. Clinical indicator testing

(1) All participants had their blood samples collected after fasting for 12 h, and serum biochemical indicators (fasting blood glucose, mean corpuscular hemoglobin, mean corpuscular volume, and serum total protein) were measured by using a fully automatic chemiluminescence analyzer.

(2) All fecal samples from the participants were stored in sterile containers at -80 °C. Bacterial DNA was extracted from the samples by using a magnetic bead method. DNA quality was assessed by 0.8% agarose gel electrophoresis, and the absorbance ratio at 260 nm and 280 nm was measured by spectrophotometry. Shotgun metagenomic sequencing was performed by using the Illumina novaseq 6000 platform. Paired-end reads of 150 bp were generated. The raw sequencing reads were subjected to quality filtering and shearing to remove the host genome. For metagenomic species annotation, MetaPhlan4 software (v4.0.6) was used for qualitative and quantitative analysis of bacterial species to obtain relative abundance at the species level.

(3) Based on the fecal metagenomic data, the gut library index of each sample was calculated by using gmwi2 software (v1.6), that is, the gut health status of the host was predicted by the gut microbiome taxonomic profile. Specifically, potential human genome contaminants (base sequences mapped to GRCh38/hg38) were first removed by using Bowtie2 software (v2.4.4). Then, overrepresented sequences in each metagenomic sample and potential Illumina universal adapter sequences were identified and extracted by using FastQC (v0.11.8). Subsequently, the adapter sequences were spliced and quality filtered by using Trimmomatic software (v0.39). Meanwhile, low-quality bases (Phred quality score < 3) before and after each read were removed, and modified reads with a length of less than 60 bp were discarded. Finally, the Lasso-penalized model was trained by using the metagenomic dataset, and predicted disease status based on binary presence/absence taxonomic features. The best performing parameters were selected in each outer loop training, and the parameters used for the GMWI2 model were finally selected.

Note: all statistical analyses in the present disclosure were completed by using R software (v4.3.3, https://www.r-project.org/). α diversity of the gut microorganisms was calculated by using the "vegan" package, and β diversity was assessed based on the Bray-Curtis distance. Differential gut microorganisms were determined by using t-tests and visualized by using the "pheatmap" package in R software. Differences in clinical indicators were analyzed by using t-tests. Histograms were generated by using GraphPad Prism (v8.0.2) statistical software, and values are expressed as mean ± SEM. The significance of differences was calculated by using t -tests; *P < 0.05, **P < 0.01, ***P < 0.001. Correlation analyses were based on the Spearman rank correlation coefficient and visualized by using the "pheatmap" package in R software.

4. Result

(1) Improvement of blood indicators: the test results of serum biochemical indicators are as shown in FIG. 3. It may be seen that the serum biochemical indicators of the subjects were healthier after taking Lactiplantibacillus plantarum GOLDGUT-HNU082 than those before taking. Specifically, the reference range of fasting blood glucose is 3.9 ~ 7.8 mmol/L. The results show that for volunteers within the reference range, the fasting blood glucose results do not fluctuate much, while for volunteers with high fasting blood glucose, blood glucose is significantly reduced after taking Lactiplantibacillus plantarum GOLDGUT-HNU082, reaching or approaching normal values. The serum total protein is an indicator related to liver function, and its reference range is 60 ~ 80 g/L. The results show that the serum total protein of the volunteers is overall high or at the high end of the normal range, and the serum total protein is significantly reduced after taking Lactiplantibacillus plantarum GOLDGUT-HNU082. The reference range of mean corpuscular hemoglobin is 27 ~ 34 pg, and for volunteers within the reference range, the results do not fluctuate much, while for volunteers with low levels of this indicator, the mean corpuscular hemoglobin increases after taking Lactiplantibacillus plantarum GOLDGUT-HNU082 and approaches normal values. The reference range of mean corpuscular volume is 80 ~ 100fL, the mean corpuscular volume significantly increased within the normal range after taking Lactiplantibacillus plantarum GOLDGUT-HNU082. Taking Lactiplantibacillus plantarum GOLDGUT-HNU082 has a reducing effect in subjects with high fasting blood glucose levels and the effect of improving liver function in subjects.

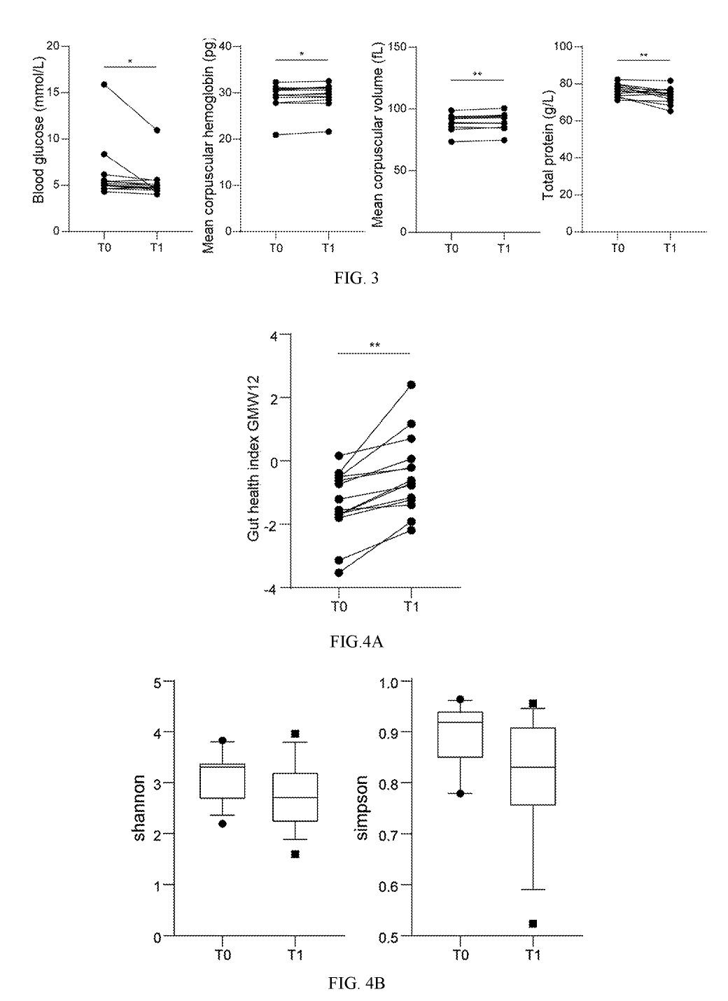

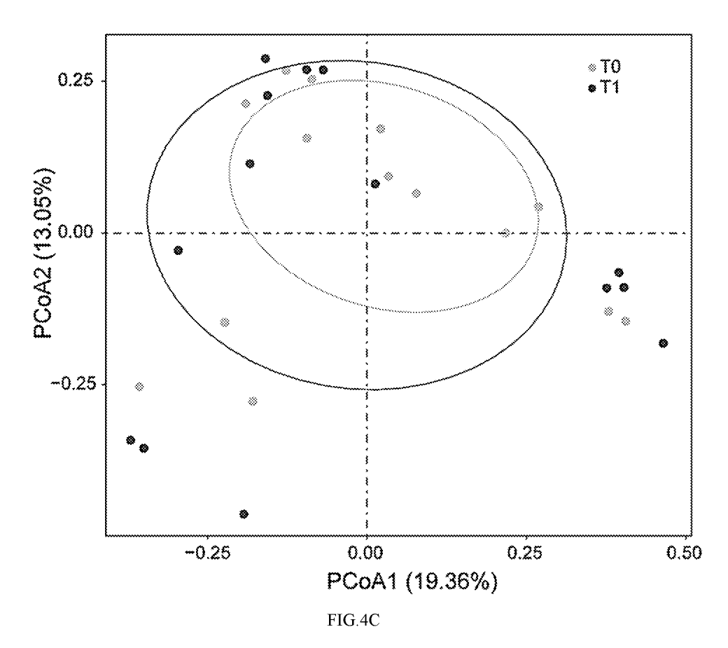

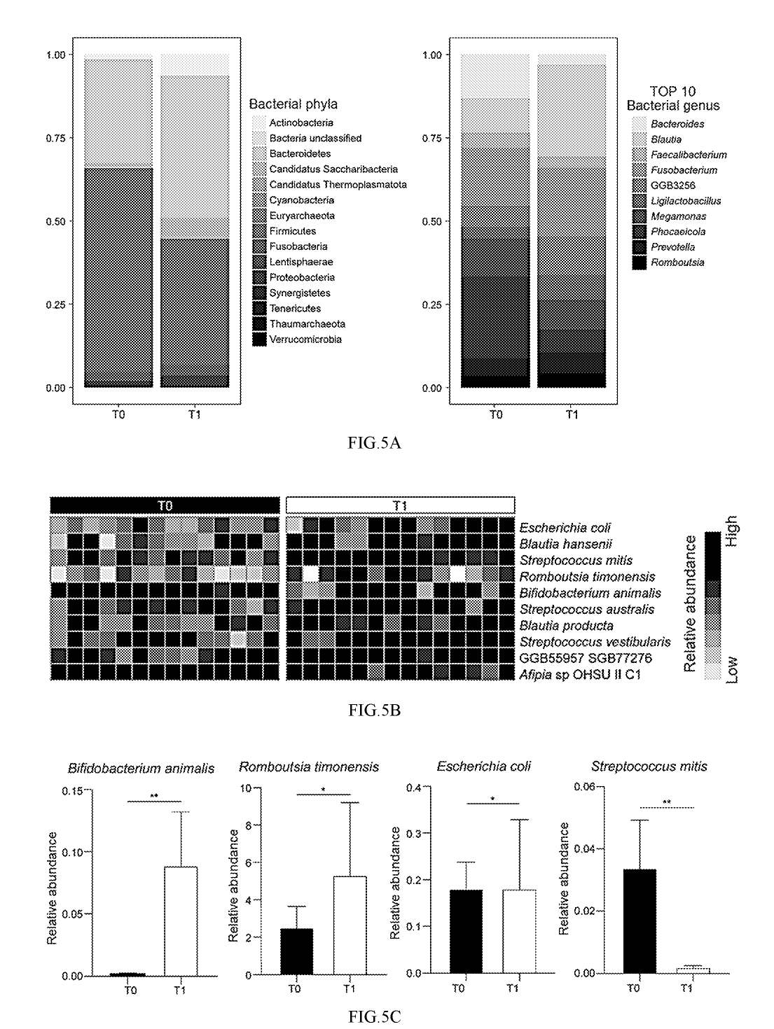

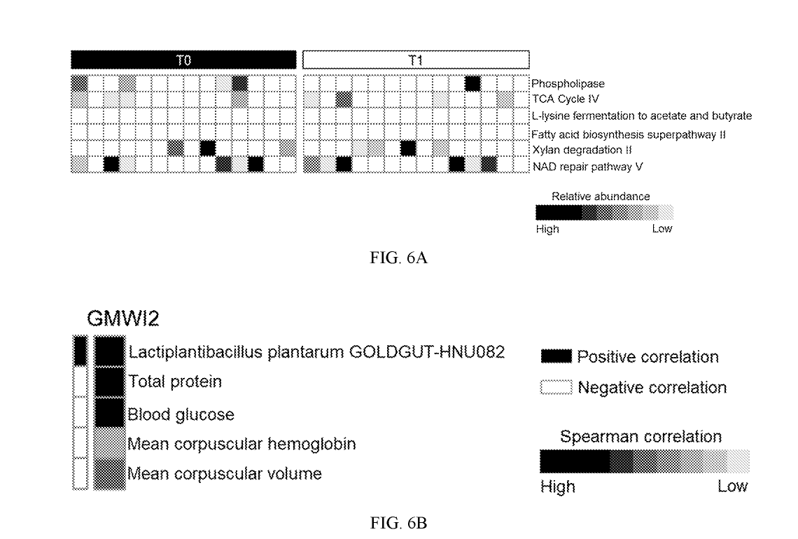

(2) Improvement of gut microbiota: although the results of α and β diversity analysis showed that the richness and structure of the gut microbial community did not change significantly after supplementation with Lactiplantibacillus plantarum GOLDGUT-HNU082 (see FIGS. 4B and 4C), it is found, by comparing the abundance of the bacterial phyla and the top 10 most abundant bacterial genera in the total gut microorganisms before and after supplementation with Lactiplantibacillus plantarum GOLDGUT-HNU082 (see FIG. 5A), that the abundance of phylum level bacterial communities reported to be beneficial to the human body, including Actinobacteria, Bacteroidetes, and Candidatus Saccharibacteria, increased, while the abundance of Cyanobacteria, Firmicutes, and Synergistetes decreased. In the above, Actinobacteria is known to produce a variety of secondary metabolites, including antibiotics, anticancer agents, and immunosuppressants; Bacteroidetes is considered to be the main degrader of polysaccharides; and Candidatus Saccharibacteria has been found to have immunomodulatory activity. At the genus level, the abundance of Blautia, Fusobacterium, GGB3256, Ligilactobacillus, Prevotella and Romboutsia increased, while the abundance of Bacteroides, Faecalibacterium, Megamonas and Phocaeicola decreased. Studies have shown that among the upregulated bacterial genera, Blautia has the ability to regulate host health and alleviate metabolic syndrome, and has probiotic properties; Ligilactobacillus murinus is widely involved in host intestinal metabolism and immune activities; Romboutsia may improve obesity by regulating gut microbial community and lipid metabolism; and among the downregulated bacterial genera, Megamonas is enriched in obese subjects. On this basis, we screened out the differential bacterial species before and after supplementation with Lactiplantibacillus plantarum GOLDGUT-HNU082, and found that beneficial bacteria in the human gut, such as Bifidobacterium animalis and Romboutsia timonensis were significantly enriched (see FIGS. 5B and 5C). In addition, the relative abundance of some harmful species, such as Escherichia coli and Streptococcus mitis, decreased significantly after supplementation with Lactiplantibacillus plantarum GOLDGUT-HNU082. Moreover, we screened metabolic pathways associated with differential bacteria and found that the metabolic pathways related to short-chain fatty acid production (L-lysine fermentation to acetate and butyrate), as well as pathways related to glycolipid metabolism (phospholipases, TCA cycle IV, fatty acid biosynthesis superpathway II, xylan degradation II, and NAD repair pathway V) were enriched after supplementation with Lactiplantibacillus plantarum GOLDGUT-HNU082 (see FIG. 6A).

(3) Improvement of human gut health: to assess human gut health, we used GMWI2 to illustrate human gut health condition and quantify the balance between health-related microorganisms and disease-related microorganisms. GMWI is a reliable and biologically interpretable health status predictor, which is based on the gut microbiome taxonomic profiling, and the higher value thereof indicates better human health condition. The results showed that the GMWI2 of the population increased significantly by 54.14% after supplementation with Lactiplantibacillus plantarum GOLDGUT-HNU082 (see FIG. 4A).

(4) GMWI2 being correlated with gut microbiota and clinical indicators: we quantified the interaction between Lactiplantibacillus plantarum GOLDGUT-HNU082 in the subjects' guts, clinical indicators, and GMWI2 by using their Spearman correlation coefficients. Correlation analysis shows that Lactiplantibacillus plantarum GOLDGUT-HNU082 is positively correlated with GMWI2, and the GMWI2 index is negatively correlated with the subjects' blood glucose levels, total protein levels, and red blood cell levels. The color is proportional to the strength of the correlation (see FIG. 6B).

The various embodiments in this specification are described in a progressive manner, and each embodiment focuses on the differences from other embodiments. The same or similar parts between the various embodiments may be referenced to each other.

The above description of the disclosed embodiments is intended to enable one skilled in the art to implement or use the present disclosure. Various modifications to these embodiments will be apparent to one skilled in the art, and the general principles defined herein may be implemented in other embodiments without departing from the spirit or scope of the present disclosure. Therefore, the present disclosure is not limited to the embodiments shown herein but is intended to conform to the widest scope consistent with the principles and novel features disclosed herein.

1. A method for improving gut health, comprising administering Lactiplantibacillus plantarum GOLDGUT-HNU082 to a subject, wherein a deposit number of the Lactiplantibacillus plantarum GOLDGUT-HNU082 is CGMCC NO. 33075.

2. A method for preparing a bacterial agent for improving gut health, a product for increasing GMWI2, a product for lowering blood glucose, or a product for reducing serum total protein, comprising using Lactiplantibacillus plantarum GOLDGUT-HNU082 to prepare the bacterial agent for improving gut health, the product for increasing GMWI2, the product for lowering blood glucose, or the product for reducing serum total protein, wherein a deposit number of the Lactiplantibacillus plantarum GOLDGUT-HNU082 is CGMCC NO. 33075.

3. A method for preparing a product for improving gut health, comprising using a bacterial agent containing Lactiplantibacillus plantarum GOLDGUT-HNU082 to prepare the product for improving gut health, wherein a deposit number of the Lactiplantibacillus plantarum GOLDGUT-HNU082 is CGMCC NO. 33075.
